(19) United States
(12) Patent Application Publication
(10) Pub. No.: US 2009/0142331 A1
(43) Pub. Date: Jun. 4, 2009

(54) Method for the Production of Bio-Active Substances from the Novel Actinomycete TAXA MAR3A, MAR3B And MAR4 Belonging to the Family Treptomycetaceae

(76) Inventors: William H. Fenical, Del Mar, CA (US); Paul R. Jensen, San Diego, CA (US)

Correspondence Address: DLA PIPER LLP (US), 4365 EXECUTIVE DRIVE, SUITE 1100, SAN DIEGO, CA, 92121-2133, US

(21) Appl. No.: 11/597,963

(22) PCT Filed: Jun. 21, 2004
(86) PCT No.: PCT/US04/19928

§ 371 Date: Aug. 15, 2007

(51) Int. Cl. A61K 35/00; C12N 1/20; C07H 21/04; C12N 15/63; C12Q 1/02; G06F 19/00; G01N 33/48; C12Q 1/68

(52) U.S. Cl. 424/115; 435/252.1; 536/23.7; 435/320.1; 435/29; 702/20; 435/6

(57) ABSTRACT

This invention provides a method for the isolation and identification of marine actinomycetes tentatively named MAR3A and MAR3B and the use of these groups as a source of new biologically active compositions including pharmaceuticals. The method includes specifics about where these microorganisms occur and their culture requirements. It also provides information describing characteristic DNA sequences that are used to identify members of this group and information demonstrating that members produce metabolites with significant activities in pharmaceutically relevant bioassays.

This invention was made in part with government support under Grant No. CA44848 awarded by the National Cancer Institute and the University of California Industry-University Cooperative Research Program (IUCRP) Grant No. 10102. The government has certain rights in this invention.

### BACKGROUND OF THE INVENTION

1. Field of the Invention

The invention relates generally to methods to isolate and identify new actinomycete taxa and, more specifically, marine forms. The methods described herein will allow these organisms to be studied as a source of new biologically active secondary metabolites. These metabolites will have potential utility as new medicines, agrichemicals, immunomodifiers, enzymes, enzyme inhibitors and for other natural product applications.

2. Background Information

As of 1988, approximately two thirds of the known, naturally derived antibiotics, including many pharmaceuticals in current clinical use, were discovered as fermentation products from cultured actinomycetes (Okami and Hotta, 1988). Although the positive impact of actinomycete products on human health is clear, there is a perception that 50 years of intensive research by the pharmaceutical industry has exhausted the supply of compounds that can be discovered from this group. This perception has been a driving force behind the recent shift away from natural products as a source of small molecule therapeutics towards other drug discovery platforms including high throughput combinatorial synthesis and rational drug design (Blondelle and Houghten, 1996; Bull et al., 2000; Wijkmans and Beckett, 2002).

Historically, actinomycetes are best known as soil bacteria and were generally believed to occur in the ocean largely as dormant spores that were washed into the sea (Goodfellow and Haynes, 1984). Despite evidence to suggest that this may not be the case (Jensen et al., 1991; Moran et al., 1995; Takizawa et al., 1993; Helmke and Weyland, 1984, Colquhoun et al., 1998), the distributions and ecological roles of actinomycetes in the marine environment, and the extent to which obligate marine species occur, has remained an unresolved issue in marine microbiology.

Recently, the present inventors have reported the cultivation from marine sediments of a major new group of marine actinomycetes (originally called MAR1) for which the generic epithet “Salinospora” was proposed (Mincer et al., 2002). The systematics of this taxon has now been studied in more detail and two species, S. arenicola and S. tropicalis are in the process of being described. Large numbers of “Salinospora” strains have been recovered from sediments collected from the sub-tropical Atlantic, the Red Sea, and the Sea of Cortez suggesting a pan-tropical distribution. To date, all “Salinospora” strains obtained in culture have required seawater for growth, indicating a high level of marine adaptation, and the taxon, overall, has proven to be a productive source of structurally unique and biologically active secondary metabolites (Feling et al., 2003). Thus, there is mounting evidence that marine actinomycetes represent an autochthanous yet little understood component of the sediment microbial community as well as a useful resource for pharmaceutical discovery.

It has long been known that actinomycetes can be recovered from marine sediments (Weyland, 1969) raising the possibility that these bacteria, like their terrestrial counterparts in soils, play important roles in the decomposition of recalcitrant organic matter in the sea floor. More recently, marine-derived actinomycetes have become recognized as a source of novel antibiotics and anticancer agents (Faulkner, 2002 and references cited therein) suggesting that they also represent a new resource for natural product drug discovery (Jensen and Fenical, 2000; Bull et al., 2000). For the former to be correct, actinomycetes must be metabolically active in the marine environment. For the later to be correct, this activity must lead to the production of compounds that are not observed from terrestrial strains. Thus, to understand the importance of marine-derived actinomycetes in ecological terms and as a resource for biotechnology, the extent to which they are metabolically active in the ocean, the degree to which they display specific marine adaptations, and the extent to which these adaptations have affected secondary metabolite production must be determined. Although evidence has been presented for the existence of indigenous marine actinomycete populations (Jensen et al., 1991; Colquhoun et al., 1998) and for in situ metabolic activity (Moran et al., 1995), the extent to which marine-adapted actinomycetes are phylogenetically unique from their terrestrial relatives is largely unknown.

A recent culture-independent study of actinobacterial diversity revealed the presence of numerous new phylotypes including many clones that were most closely related to Streptomyces sp. (Stach et al., 2003). Many of these clones possessed ≦97% identity with previously cultured species suggesting the existence of multiple new genera. NCBI BLAST (blastn) searches of the new phylotypes cultured as part of the present invention revealed none of the accession numbers reported by Stach and co-workers indicating that a great deal of marine actinomycete diversity remains to be cultured from marine sediments. Although significant progress has been made recently in the development of innovative techniques for the cultivation of marine bacteria (e.g., Rappé et al., 2002), it is clear that continued improvements in taxa-specific cultivation methods have the potential to yield significant new marine actinomycete diversity.

Streptomyces were found to be metabolically active in marine sediments (Moran et al., 1995) making it possible that a lack of genetic mixing with terrestrial strains coupled with the adaptations required for survival in the marine environment have led to the evolution of obligate marine taxa within the Streptomycetaceae and other actinomycete families. Thus, there is a strong need for new groups of actinomycetes to be evaluated if new and better drugs are to be discovered.

### SUMMARY OF THE INVENTION

The present invention relates to the isolation and identification of groups of marine actinomycetes and the use of these groups as a source of new biologically active agents, including pharmaceuticals. The methods include disclosure of where these microorganisms can be obtained and their culture requirements. It also provides specific information describing characteristic DNA sequences that are used to identify members of this group and discloses that members of this group produce metabolites with significant activities in pharmaceutically relevant bioassays.

In the present invention, actinomycetes belonging to the MAR3A and MAR3B group were cultivated from samples collected from various locations in Guam and around San Diego, and include, but are not limited to, members found in other locations.

In one embodiment, a bacterial preparation, which includes one or more isolated and purified strain(s) of a microorganism that produces one or more bioactive compositions is envisaged, where the strain is cultured in a nutrient medium usually, but not limited to, containing seawater or added salts, where such a strain(s) produces at least one secondary metabolite. In a related aspect, the secondary metabolites may have anticancer and/or antibiotic activity.

In another embodiment, bacterial preparations include one or more isolated and purified strain(s) of marine actinomycetes are envisaged where the bioactive compositions containing secondary metabolites are produced by cultivating the strains on media/compositions as described above.

Such organisms may include actinomycetes that fall within the MAR3A and/or MAR3B phylotypes within the Streptomycetaceae based on SSU rRNA gene sequence analysis using, for example, methods of sequencing and tree construction such as PAUP.

In one embodiment, isolated bacteria having defined 16S rRNA are disclosed. Such rRNA may be transcribed from a nucleotide sequence that includes the DNA sequence as shown in SEQ ID NO: 7, SEQ ID NO: 8, SEQ ID NO: 9, SEQ ID NO: 10, SEQ ID NO: 11, SEQ ID NO: 12 or SEQ ID NO: 13 of the present invention.

In a related aspect, such isolated bacteria may possess the following morphological characteristics, including but not limited to, positive gram staining, filamentous hyphae, mycelium forming and/or forming leathery colonies that adhere to agar surfaces.

In one embodiment, novel strains of marine actinomycetes are described. In a related aspect, on Jun. 9, 2004, strains CNQ687 and CNQ251 were deposited at the American Type Culture Collection (ATCC), Manassas, Va., USA, under the terms of the Budapest Treaty on the International Recognition of the Deposit of Microorganisms for the Purposes of Patent Procedure and Regulations thereunder (Budapest Treaty), and are thus maintained and made available according to the terms of the Budapest Treaty. Availability of such strains is not to be construed as a license to practice the invention in contravention of the rights granted under the authority of any government in accordance with its patent laws.

In a further related aspect, the deposited strains have been assigned the indicated ATCC deposit numbers:

CNQ687: ATCC No. ______, and CNQ251: ATCC No. ______.

In another embodiment, a method is disclosed for producing and isolating bioactive compositions having antibiotic and/or anticancer properties from one or more strains of actinomycetes, belonging to the MAR3A and MAR3B groups. Strains belonging to these groups will be cultured in a medium that may contain, but is not limited to, a certain amount of seawater or salts and various nutrients such as yeast extract, peptone, starch and glucose. Strains may be cultured in liquid media or on solid surfaces (e.g., agar).

Further, the resulting cultured strains may be extracted with an absorbent resin (e.g., but not limited to, XAD-7) and subsequently eluted with a polar organic solvent such as acetone. The cultures may also be extracted with an organic solvent (e.g., ethyl acetate) or freeze-dried and extracted with an organic solvent (e.g., methanol). In a related aspect, the resulting solution is evaporated and the remaining solute is solubilized in a chaotropic agent (e.g., DMSO), where the solubilized residue contains the bioactive composition.

In another embodiment, a bioactive composition is isolated from extracts of cultured strains of marine actinomycetes belonging to the MAR3A and/or MAR3B groups using, for example, column chromatography, HPLC, counter-current chromatography, or any methods familiar to one skilled in the art. Once in pure form, the structures of these compounds can be resolved by NMR and other spectral methods.

In a related aspect, the secondary metabolites may be assayed for pharmaceutical, agrichemical, or other biotechnological related activities. For example, isolated bioactive compositions are effective against methylicillin-resistant Staphylococcus aureus (MRSA) and HCT-116 human carcinoma cells.

In one embodiment, isolated nucleic acids including SEQ ID NO: 7, SEQ ID NO: 8, SEQ ID NO: 9, SEQ ID NO: 10, SEQ ID NO:11, SEQ ID NO: 12 or SEQ ID NO: 13 are envisaged, where such nucleic acids may be DNA or RNA. In a related aspect, such nucleic acids may include linkage to a vector. In a further aspect, a host cell may harbor the vector containing the nucleic acids as described above.

In another embodiment, a method for identifying strains that belong to the MAR3A/MAR3B clades is envisaged, including performing BLAST comparison searches of a public or private databases using, for example, default parameters, where 16S rRNA sequences of the bacterial strains are compared via a sequence comparison interface of the database. Further, text files can be generated which comprise a subset of resulting hits, where the text files are analyzed on a public or private ribosomal database using a sequence matching interface. Moreover, sequence homologies between the subset of hits from the BLAST search and sequences for MAR3A and/or MAR3B strains of bacteria can be determined and strains belonging to the MAR3A and/or MAR3B group based on the similarity between sequence alignments that are generated by the sequence matching interface of the ribosomal database can be identified.

The present invention also envisages methods for drug discovery which includes growing a strain of actinomycete, such as MAR3A or MAR3B, collecting an actinomycete extract or conditioned growth media and analyzing the extract or media for pharmaceutical activity.

In another embodiment, methods for treating cancer and infections using bioactive compositions are disclosed. In a related aspect, such bioactive compositions embrace pharmaceutical compositions which include a pharmaceutically acceptable carrier.

Exemplary methods and systems according to this invention are described in greater detail below.

### BRIEF DESCRIPTION OF THE DRAWINGS

FIG. 1 shows phylogenetic relationships among nearly full SSU rRNA gene sequences of cultured marine actinomycetes belonging to the MAR3A and MAR3B actinomycetes and closely related sequences obtained from an NCBI BLAST (blastn) search. The tree was constructed using the neighbor-joining method with the percentage of bootstrap replicates (1000 re-samplings) supporting the proposed branching order shown at the relevant nodes (values below 55%). Bifidobacterium angulatum and Propionibacterium propionicus were used as outgroups.

FIG. 2 shows complete DNA sequences encoding 16S rRNAs for MAR3A and MAR3B. MAR3A: CNQ687 (SEQ ID NO: 7), CNQ698 (SEQ ID NO: 8), CNQ719 (SEQ ID NO: 9), and CNQ530 (SEQ ID NO: 11); MAR3B: CNR530 (SEQ ID NO: 10), CNQ265 (SEQ ID NO: 12), and CNQ662 (SEQ ID NO: 13).

### DETAILED DESCRIPTION OF THE INVENTION

Before the present methods are described, it is understood that this invention is not limited to the particular methodology, protocols, cell lines, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a subject” includes a plurality of such subjects, reference to a “nucleic acid” is a reference to one or more nucleic acids and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the methods, devices, and materials are now described. All publications mentioned herein are incorporated herein by reference for the purpose of describing and disclosing the compounds, and methodologies which are reported in the publications which might be used in connection with the invention. Nothing herein is to be construed as an admission that the invention is not entitled to antedate such disclosure by virtue of prior invention.

As used herein, “bioactive composition,” means a combination consisting essentially of secondary metabolites produced by microbes that increase the ability of such organisms to survive and proliferate, where these metabolites are generally thought to be nonessential for growth of the organism and are synthesized with the aid of genes involved in intermediary metabolism. The largest class of secondary metabolites are the polyketides, which include a broad range of antibiotics, immunosuppressants and anticancer agents (e.g., erythromycin, tetracycline, FK506, doxorubicin, etc.).

As used herein, “clade” (i.e., a monophyletic group of organisms) means a group of organisms that all share a common ancestor as defined by gene sequence analyses. In a related aspect, monophyletic is a term applied to a group of organisms which includes the most recent common ancestor of all of its members and all of the descendants of that most recent common ancestor. For example, a monophyletic group is called a clade.

As used herein, “genus” means a category of biological classification ranking between the family and the species, comprising structurally or phylogenetically related species or an isolated species exhibiting unusual differentiation. In a related aspect, “species” herein means a biological classification comprising related organisms that share common characteristics.

As used herein, “strain” means an individual organism that is representative of a group of organisms that share common ancestry with clear-cut physiological and in some cases morphological distinctions.

A used herein, “taxon” means any named group of organisms, not necessarily a clade; a taxon may be designated by a Latin name or by a letter, number, or any other symbol.

As used herein, Family Streptomycetaceae includes at least three genera of filamentous bacteria of the Order Actinomycetales, consisting of higher bacteria that comprise several hundred different species typically occurring in soil and water, but may also be found as parasites on plants and animals. For example, Streptomyces is a genus of this Family.

Isolated nucleic acids specific for marine actinomycetes are also provided. An example of such nucleic acids comprises the nucleotide sequences defined in the Sequence Listing as SEQ ID NO: 7, SEQ ID NO: 8, SEQ ID NO: 9, SEQ ID NO: 10, SEQ ID NO: 11, SEQ ID NO: 12 and SEQ ID NO: 13 is provided. These sequences are DNA equivalents of unique 16S ribosomal RNAs of MAR3A and MAR3B groups. Isolated double-stranded nucleic acids encoding the 16S rRNA are also used to define the MAR3A and MAR3B groups and are also provided. Such nucleic acids can be used for identification of MAR3A and MAR3B and phylotypes which synthesize bioactive compositions, for example, by polymerase chain reaction or sequence comparison.

In one embodiment, an isolated nucleic acid that selectively hybridizes under stringent conditions with and has at least 98.4%, 98.5%, 98.6%, 98.7%, 98.8%, 98.9% or 99% sequence complementarity with a segment of the strand of the 16S rDNA of MAR3A or at least 97.5%, 97.6%, 97.7%, 97.8%, 97.9%, 98% or 99% sequence complementarity with a segment of the strand of the 16S rDNA of MAR3B to which it hybridizes is also provided. As used herein, to describe nucleic acids, the term “selectively hybridizes” means the same as “specific hybridization” and excludes the occasional randomly hybridizing nucleic acids as well as nucleic acids that encode ribosomal RNAs from other species. The selectively hybridizing nucleic acids can be used, for example, as probes or primers for detecting the presence of an organism that has the nucleic acid to which it hybridizes. Thus, the invention provides a method of detecting MAR3A and MAR3B phylotypes, comprising detecting the presence of the selectively hybridizing nucleic acid in a sample.

The selectively hybridizing nucleic acids of the invention, for example, can have at least 97.5%, 98% and 99% complementarity with the segment and strand of the sequence to which it hybridizes, depending on the strain of interest. The nucleic acids are typically 12 to 2000 nucleotides in length. Thus, for example, the nucleic acid can be used as a probe or primer in a method for detecting the presence of bioactive composition producing MAR3A and/or MAR3B phylotypes. If used as primers, the invention provides compositions including at least two nucleic acids which selectively hybridize with different regions on the two strands of the DNA so as to amplify a desired region. For example, for the purpose of identifying MAR3A and MAR3B phylotypes, the degree of complementarity between the hybridizing nucleic acid (probe or primer) and the sequence to which it hybridizes (MAR3A and MAR3B DNA from a sample) should be at least enough to exclude hybridization with a nucleic acid from a related bacterium (e.g., other actinomycetes species). Thus, a nucleic acid that selectively hybridizes with phylotypes of MAR3A or MAR3B 16S rDNA sequences will not selectively hybridize under stringent conditions with a nucleic acid for a 16S rDNA of other phylotypes, and vice versa. The degree of complementarity required to distinguish selectively hybridizing from nonselectively hybridizing nucleic acids under stringent conditions can be readily determined for each nucleic acid by testing it for its ability to hybridize under stringent conditions with non-MAR3A or MAR3B actinomycetes bacteria.

“Stringent conditions” refers to the washing conditions used in a hybridization protocol. In general, the washing conditions should be a combination of temperature and salt concentration chosen so that the denaturation temperature is approximately 5°-20° C. below the calculated Tₘ (melting/denaturation temperature) of the hybrid under study. The temperature and salt conditions are readily determined empirically in preliminary experiments in which samples of reference DNA immobilized on filters are hybridized to the probe or coding nucleic acid of interest and then washed under conditions of different stringencies. For hybridization with a large oligonucleotide probe (e.g., 470 bp) hybridization is at 68° C. in the presence of 5×SSPE, followed by removing the non-specific hybrids by high-stringency washes of 0.1×SSPE at 68° C. as described in Sambrook et al. (1989). Hybridizations with oligonucleotide probes of 18 or fewer nucleotides in length are done at 5°-10° C. below the estimated Tₘ in 6×SSPE, then washed at the same temperature in 2×SSPE as described in Sambrook et al. (1989). The Tₘ of an oligonucleotide can be estimated by allowing 2° C. for each A or T nucleotide, and 4° C. for each G or C. An 18 nucleotide probe or primer of 50% G+C would, therefore, have an approximate Tₘ of 54° C. Thus, stringent conditions for such an 18 nucleotide probe or primer would be a Tₘ of about 54° C. and washing a salt concentration of about 0.1×SSPE, which can be modified as necessary in preliminary experiments.

It is noted that detection of the bacterium of the present invention can include consideration of characteristics other than the presence of the disclosed 16S ribosomal RNAs or DNA sequences provided herein. By considering other characteristics, such as biochemical or ultrastructural features of an isolated bacterium, it is possible to identify MAR3A or MAR3β isolates that do not have the identical 16S rRNA sequence provided in SEQ ID NO: 7, SEQ ID NO: 8, SEQ ID NO: 9, SEQ ID NO: 10, SEQ ID NO: 11, SEQ ID NO: 12 and SEQ ID NO: 13. It is expected that the 16S cistron of such an isolate can selectively hybridize with the disclosed DNA since there will be sufficient sequence similarity between the newly obtained isolate and the type strain provided herein. It is also expected that analysis of the 16S rRNA gene sequence of such an isolate will place it within a clade comprised by other MAR3A or MAR3B strains.

Sequence homology, identity or similarity may be determined by BLAST (Basic Local Alignment Search Tool) analysis using the algorithm employed by the programs blastp, blastn, blastx, tblastn and tblastx (Karlin, et al. Proc. Natl. Acad. Sci. USA 87: 2264-2268 (1990) and Altschul, S. F. J. Mol. Evol. 36: 290-300 (1993), fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff, et al. Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively.

In a related aspect, for NCBI-BLAST (National Center for Biotechnology Information (NCBI), U.S. National Library of Medicine, 8600 Rockville Pike, Bethesda, Md. 20894) “nr” means all GenBank, EMBL, DDBJ, and PDB sequences (but no EST, STS, GSS, or phase 0, 1 or 2 HTGS sequences).

In one embodiment, a method for determining whether a strain belongs to the MAR3A or MAR3B group is envisaged, where the method includes, but is not limited to, performing a standard BLAST (e.g., BLAST 2.0) search (blastn) using, for example, a publicly available database, such as that hosted by NCBI, where a nucleic acid sequence (e.g., 16S rRNA) is entered into a dialog box/browser using nr (default) value for conducting the search in the database. The data is processed and if the strain in question is related to members of the Streptomycetaceae Family, the process is continued by downloading the sequence data from the most closely related strain(s) from the BLAST search into a text file using, for example, a FASTA format.

The resulting data can then be analyzed by accessing a publicly available Ribosomal Database, such as the Ribosomal Database Project hosted by Michigan State University (Cole et al., (2003)), where an online analysis can be selected and run using a Phylip interface. A new session within the Phylip interface is implemented and edit dataset is selected. Further, the sequence data from the strain in question can be uploaded and aligned, and the most closely related sequence data from the BLAST search is compared to the sequence data from the MAR3A and MAR3B strains. In a related aspect, one or more Ribosome Database Project (RDP) neighbors can also be included.

Once the data has been uploaded and aligned, the data can be subjected to a Distance Matrix analysis. The results will indicate the similarities among the various sequences. Further, a phylogenetic tree may be generated from such data using an outgroup, preferably a strain from a related Family.

Thus, strains can be identified as belonging to the MAR3A or MAR3B groups by having, for example, greater than or equal to about 97.5%, 98% or 99% sequence similarity to members of one of these groups, being more similar to members of these groups than to any other sequence that has been publicly disclosed, and by joining a clade, as depicted in the tree, that only contains members of one of these groups. In a related aspect, strains can be identified as belonging to the MAR3A or MAR3B groups.

In another related aspect, MAR3B clade members can be identified by their possession of the 16S rRNA signature nucleotides as presented in the following table.

| | | MAR3B |
|---|---|---|
| Position | E. coli* | Streptomycetaceae |
| 145 | C | U |
| 771 | A | G |
| 808 | U | C |
| 1005 | U | A/C |

*The use of the Escherichia coli 16S rRNA gene sequence as an alignment reference has been used historically as a technique for developing the use of 16S rRNA gene sequences for rapid and unambiguous characterization of prokaryotes. The E. coli 16S rRNA gene sequence numbering system was developed to communicate to those skilled in the art exactly where areas of homology or primer regions, for example, occur. The technique regarding the use of the E. coli number system is disclosed in Lane et al., (1985), herein incorporated by reference in its entirety.

A nucleic acid of the present invention in a vector suitable for expression of the nucleic acid is also provided. The vector can be in a host suitable for expressing the nucleic acid.

There are numerous E. Coli expression vectors known to one of ordinary skill in the art useful for the expression of, for example, an antigen. Other microbial hosts suitable for use include bacilli, such as Bacillus subtilus, and other enterobacteriaceae, such as Salmonella, Serratia, and various Pseudomonas species. Additionally, yeast expression can be used. Mammalian cells permit the expression of proteins in an environment that favors important post-translational modifications such as folding and cysteine pairing, addition of complex carbohydrate structures, and secretion of active protein.

Polynucleotides encoding a variant polypeptide may include sequences that facilitate transcription (expression sequences) and translation of the coding sequences such that an encoded polypeptide product, for example, is produced. Construction of such polynucleotides is well known in the art. For example, such polynucleotides can include a promoter, a transcription termination site (polyadenylation site in eukaryotic expression hosts), a ribosome binding site, and, optionally, an enhancer for use in eukaryotic expression hosts, and, optionally, sequences necessary for replication of a vector.

The DNA sequences can be expressed in hosts after the sequences have been operably linked to, i.e., positioned to ensure the functioning of, an expression control sequence. These expression vectors are typically replicable in the host organisms either as episomes or as an integral part of the host chromosomal DNA. Commonly, expression vectors can contain selection markers, e.g., tetracycline resistance or hygromycin resistance, to permit detection and/or selection of those cells transformed with the desired DNA sequences (see, e.g., U.S. Pat. No. 4,704,362).

Having provided nucleic acids specific for marine actinomycetes MAR3A and MAR3B, the invention provides a method of detecting the presence of marine actinomycetes in a sample, comprising amplifying a DNA in the sample that is specific for marine actinomycetes and detecting the presence of amplification, indicating the presence of marine actinomycetes in the sample.

As more specifically exemplified below, a nucleic acid sequence specific for marine actinomycetes MAR3A and MAR3B can comprise nucleic acids coding for a 16S ribosomal RNA subunit. It is apparent that a skilled artisan can apply the methods described herein for detecting the 16S ribosomal RNA gene to detect other nucleic acid sequences specific for marine actinomycetes MAR3A and MAR3B. Examples of other sequences specific for the marine actinomycetes can include the genes for, urease, citrate synthase, heat shock protein, antigenic proteins and certain metabolic and synthetic enzymes. These genes can be obtained using probes based on the same gene from related species. The prospective marine actinomycetes specific gene can then be amplified and sequenced according to methods known in the art. The specificity of these sequences for marine actinomycetes MAR3A and MAR3B can be determined by conducting a computerized comparison with known sequences, catalogued in GenBank, a computerized database, using the computer program Gap of the Genetics Computer Group, (Madison, Wis.) or other available nucleic acid sequence databases, which search the catalogued sequences for similarities to the gene in question.

The nucleic acid specific for the marine actinomycetes MAR3A and MAR3B can be detected utilizing a nucleic acid amplification technique, such as polymerase chain reaction or ligase chain reaction. PCR primers which hybridize only with nucleic acids specific for the marine actinomycetes can be utilized. The presence of amplification indicates the presence of the organism. Thus, the present invention provides a method of detecting the presence of a specific marine actinomycetes by selective amplification. In yet another embodiment, a marine actinomycetes can be detected by directly hybridizing the unique sequence with a specifically or selectively hybridizing nucleic acid probe. Furthermore, the nucleotide sequence could be amplified prior to hybridization by the methods described above.

Alternatively, the nucleic acid is detected utilizing direct hybridization or by utilizing a restriction fragment length polymorphism. For example, the present invention contemplates a method of detecting the presence of a marine actinomycetes MAR3A and MAR3B, comprising ascertaining the presence of a nucleotide sequence associated with a restriction endonuclease cleavage site. In another embodiment a nucleic acid in a sample can be sequenced directly using, for example, Sanger ddNTp sequencing or 7-deaza-2′-deoxyguanosine 5′-triphosphate and compared to the corresponding sequence of other organisms.

Pharmaceutical compositions employed as a component of invention articles of manufacture can be used in the form of a solid, a solution, an emulsion, a dispersion, a micelle, a liposome, and the like, wherein the resulting composition contains one or more of the bioactive compositions described in the present invention as an active ingredient, in admixture with an organic or inorganic carrier or excipient suitable for enteral or parenteral applications. Compounds employed for use as a component of invention articles of manufacture may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used.

The present invention also provides pharmaceutical compositions comprising at least one compound capable of treating a disorder in an amount effective therefor, and a pharmaceutically acceptable vehicle or diluent. The compositions of the present invention may contain other therapeutic agents as described, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Invention pharmaceutical compositions may be administered by any suitable means, for example, orally, such as in the form of tablets, capsules, granules or powders; sublingually; buccally; parenterally, such as by subcutaneous, intravenous, intramuscular, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The term “therapeutically effective amount” means the amount of the subject compound(s) that will elicit the biological or medical response of a tissue, system, animal or human that is being sought by the researcher, veterinarian, medical doctor or other clinician.

The term “composition,” as used herein, is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound(s) is included in an amount sufficient to produce the desired effect upon the process or condition of diseases.

The pharmaceutical compositions containing the active ingredient(s) may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

In the treatment of a subject where cells are targeted for modulation, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The invention will now be described in greater detail by reference to the following non-limiting examples. The following examples are intended to illustrate but not limit the invention.

### EXAMPLES

### Example 1

### Sample Collection and Bacterial Isolation

Two hundred and seventy-five marine samples were collected around the island of Guam in the Southern reaches of the Northern Mariana Islands from 10-26 Jan. 2002. The samples consisted of 227 sediments (ranging from fine muds to small rocks), 33 algae, and 15 sponges. Algae, invertebrates, and shallow sediments were collected by divers from depths of 1-20 m. The remaining sediments were collected using a modified, surface deployed sediment sampler (Kahlsico, El Cajon, Calif. model #214WA 110) to depths of 570 m. All samples were processed within a few hours of collection at the marine laboratory of the University of Guam using a variety of techniques designed to reduce the numbers of Gram-negative bacteria and to enrich for slow-growing, spore-forming actinomycetes. Samples were processed and inoculated onto various agar media using one or more methods as described below. For example, all algal samples were processed using grinding with mortar and pestle, while all sponges were processed using freeze/thawing wet sediment, which was subsequently diluted, and then incubated at room temperature for 48 hours prior to inoculation on the surface of an agar plate.

Sediment was dried overnight in a laminar flow hood and, when clumping occurred, ground lightly with an alcohol-sterilized mortar and pestle. An autoclaved foam plug (2 cm dia.) was pressed onto the sediment and then repeatedly onto the surface of an agar plate in a clockwise direction creating a serial dilution effect.

Processed samples were inoculated as described above onto the surface of one of the following agar media.

Medium 1 (AMM). Eighteen grams agar, 10 g starch, 4 g yeast extract, 2 g peptone.

Medium 2 (SMC). Eight grams noble (purified) agar, 500 mg mannitol, 100 mg casamino acids, nystatin (50 μg/ml).

Medium 3 (SPC). Eighteen grams agar, polymixin B sulfate (5 μg/ml).

Medium 4 (SRC). Eighteen grams agar, rifampacin (5 μg/ml).

All media were prepared with 100% natural seawater and contained the antifungal agents cycloheximide (100 μg/ml) and, when listed, nystatin (50 μg/ml). Media were prepared by methods well known in the art.

Inoculated Petri dishes were incubated at room temperature (ca. 28° C.) and monitored periodically over three months for actinomycete growth. Actinomycetes were recognized by the presence of filamentous hyphae, and/or by the formation of tough, leathery colonies that adhered to the agar surface. All pure strains were grown in liquid culture (medium 1 without agar) and cryopreserved at −80° C. in 10% glycerol.

### Example 2

DNA extraction, PCR amplification, and phylogenetic analyses.

Genomic DNA template was prepared as previously described (Mincer et al., 2002) following a method modified from Marmur (1961). The small subunit (SSU) rRNA gene was PCR amplified using the primers FC27 (5′-AGAGTTTGATCCTGGCTCAG-3′) (SEQ ID NO: 1) and RC1492 (5′-TACGGCTACCTTGTTACGACTT-3′) (SEQ ID NO: 2) and the products purified using a Qiagen QIAquick PCR cleanup kit following the manufacturer's protocols (Qiagen Inc., Chatsworth, Calif.). PCR products were quantified and submitted to the UCSD Cancer Center DNA Sequencing Shared Resource for partial sequencing (3100 Genetic Analyzer, PE-Applied Biosystems, USA) using the primer FC27. Partial SSU rRNA gene sequences (ca. 0.6 kb) were aligned using the Ribosomal Database Project (RDPII) Phylip interface (Michigan State University, East Lansing, Mich., release number 8.1; Cole et al., 2003). Aligned sequences and related sequences obtained from an NCBI BLAST (blastn) search were imported into MacClade (version 4.03, Maddison and Maddison, 2001) and further aligned by hand. Neighbor-joining trees were created using PAUP (version 4.0b10, Swofford, 2002) and phylogenetically diverse strains selected for nearly full SSU rRNA gene sequencing of both top and bottom strands using the additional forward primers F514 (5′-GTGCCAGCAGCCGCGGTAA-3′) (SEQ ID NO: 3) and F1114 (5′-GCAACGAGCGCAACCC-3′) (SEQ ID NO: 4) and the reverse primers R530 (5′-CCGCGGCTGCTGGCACGTA-3′) (SEQ ID NO: 5) and R936 (5′-GTGCGGGCCCCCGTCAATT-3′) (SEQ ID NO: 6).

Upper and lower strand contigs were assembled in MacClade and base calling ambiguities resolved by reviewing the sequencing chromatograms in Editview (version 1.0.1, Applied Biosystems, Foster City, Calif.). The resulting ca. 1.5 kb sequences, along with related sequences obtained from an NCBI BLAST (blastn) search, were imported into Clustal X (version 1.8, Thompson et al., 1997) where multiple alignments were performed using the default alignment parameters. Aligned sequences were imported into MacClade where manual refinements were made and ambiguous nucleotides masked resulting in the inclusion of 1476 nucleotide positions in the phylogenetic analyses. Phylogenetic neighbor joining and maximum parsimony analyses were performed using PAUP (4.0b10, Sinauer Assoc., Inc., Sunderland, Mass.). Similarity values were generated using the RDPII Phylip interface distance matrix function following the Kimura 2-parameter method.

### Nucleotide Sequence Accession Numbers.

Examples of nucleotide sequence data for MAR3A and MAR3B strains have been deposited in GenBank under accession numbers AY464540-AY464545.

### Phylogenetic Diversity.

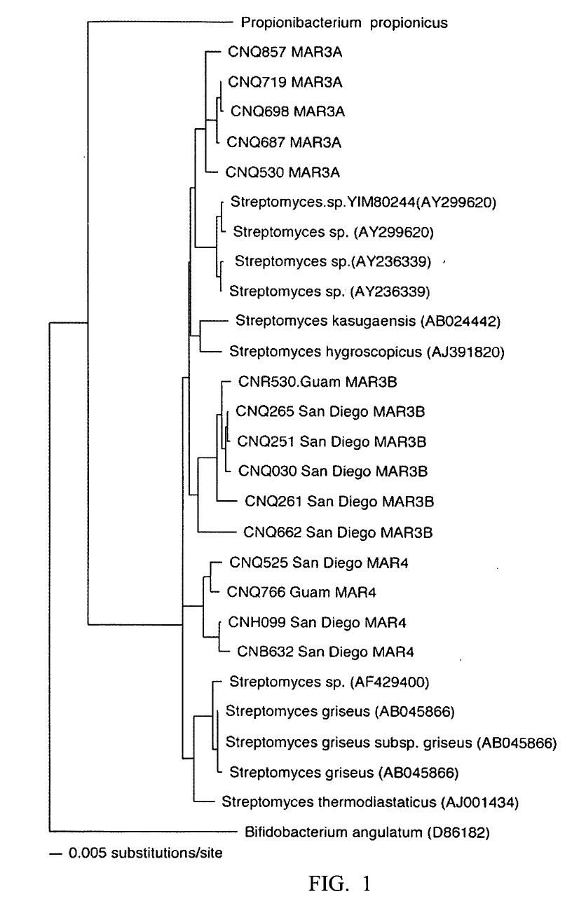

The nearly full 16S rRNA gene sequences of various MAR3A and MAR3B strains shown in FIG. 1 clearly demonstrates the phylogenetic coherency of these two groups and their separation from the most closely related organisms for which sequence data is available. These groups all fall within the family Streptomycetaceae and appear to represent two new genera. As more sequence data is obtained in the future, the topology of this tree will change and the MAR3A and MAR3B groups may be subdivided into additional groups. However, these smaller groups will still be defined by the clades created by their sequence relatedness.

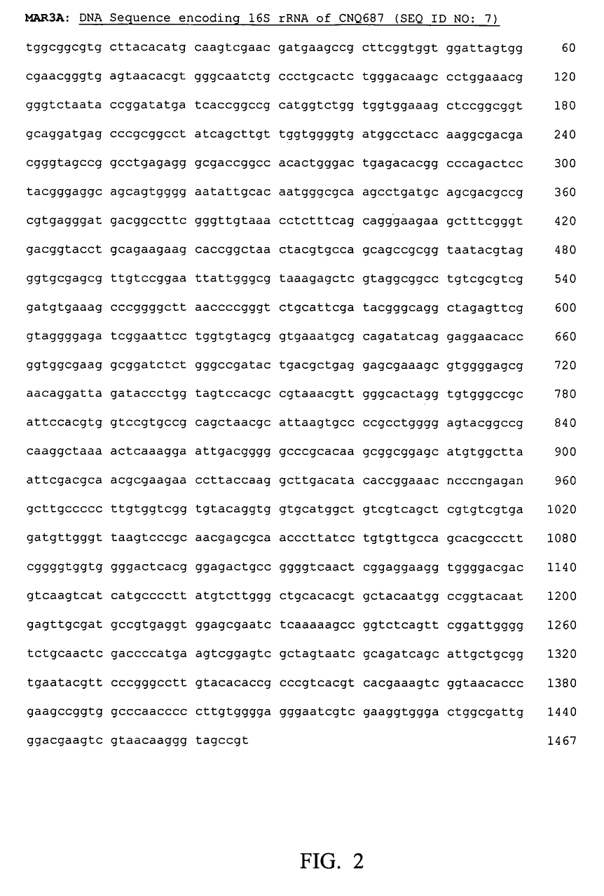

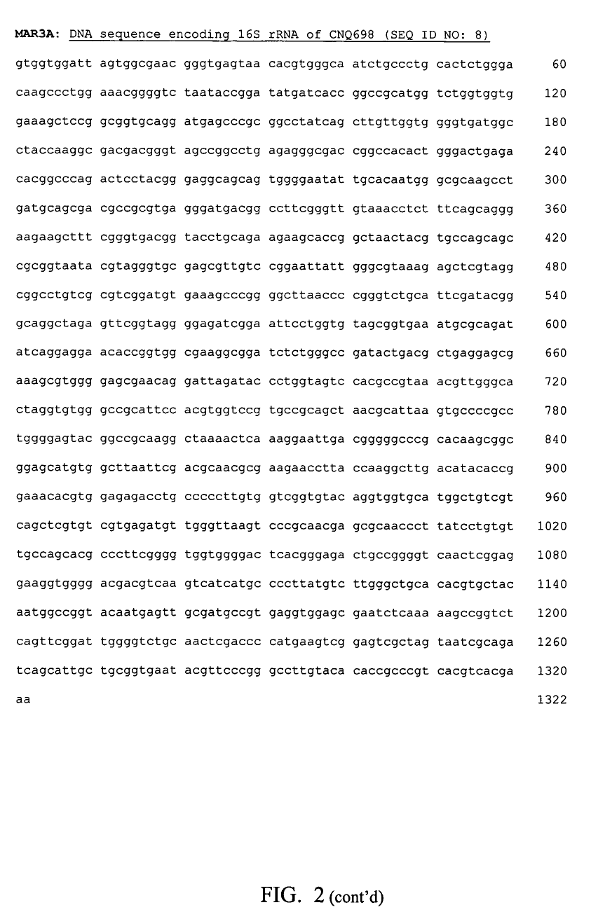

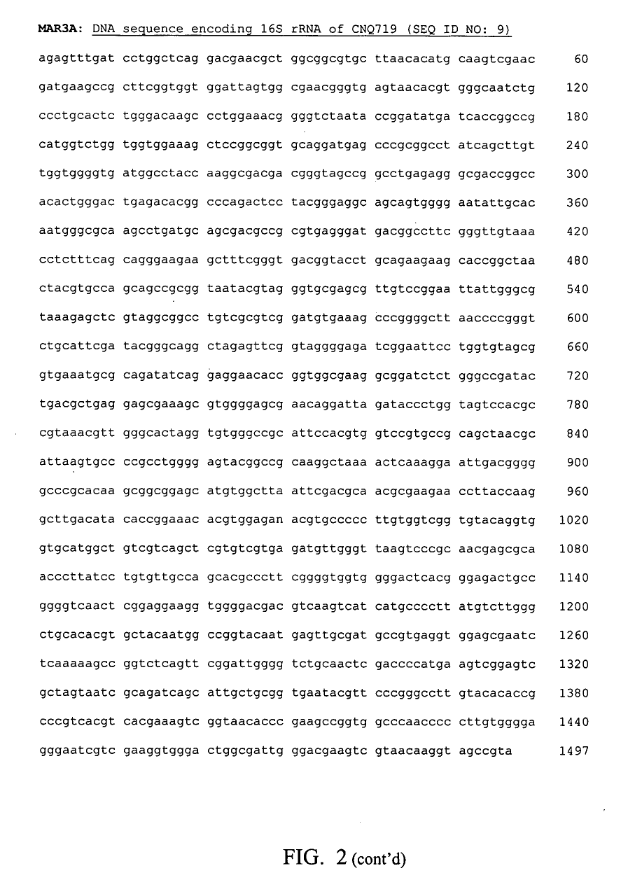

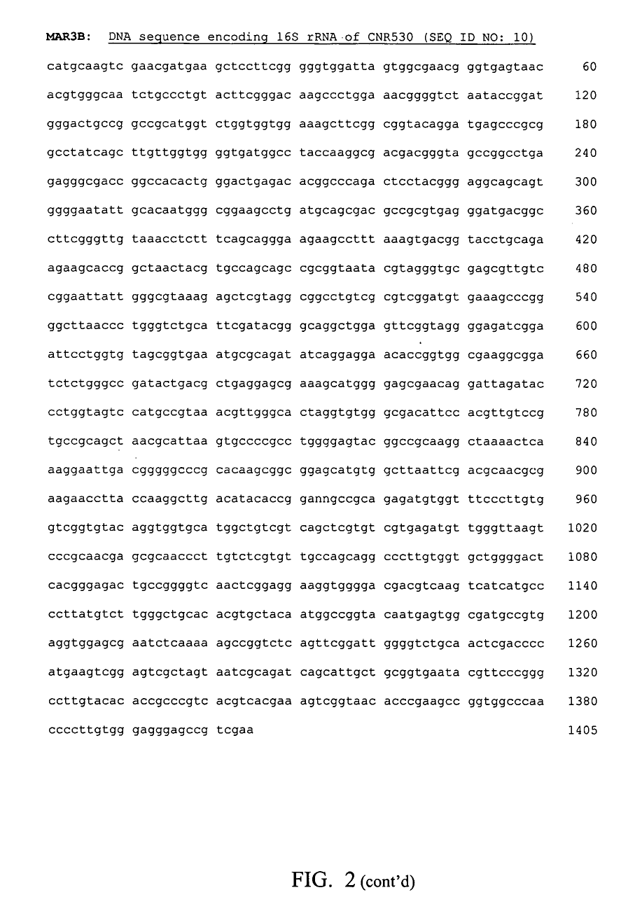

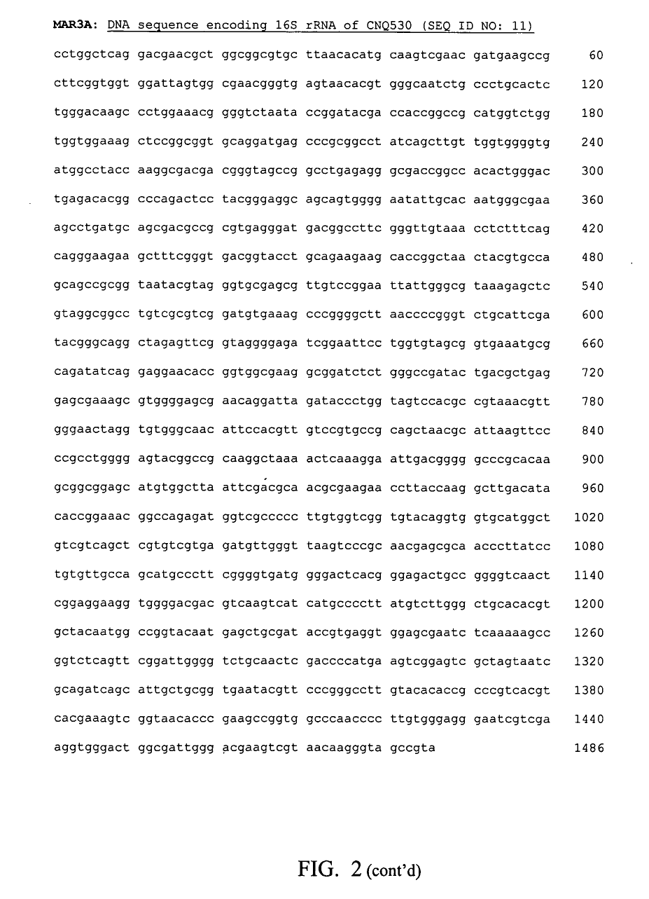

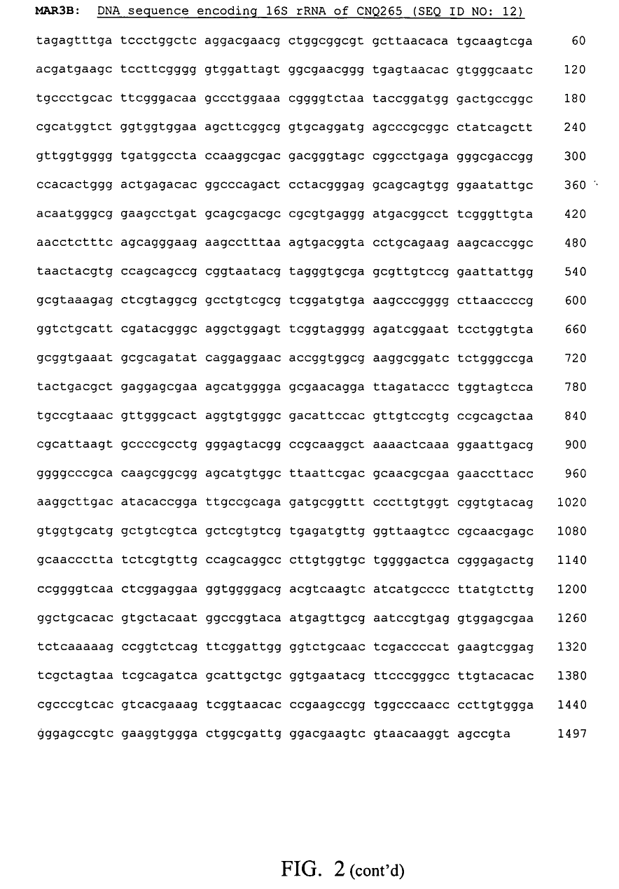

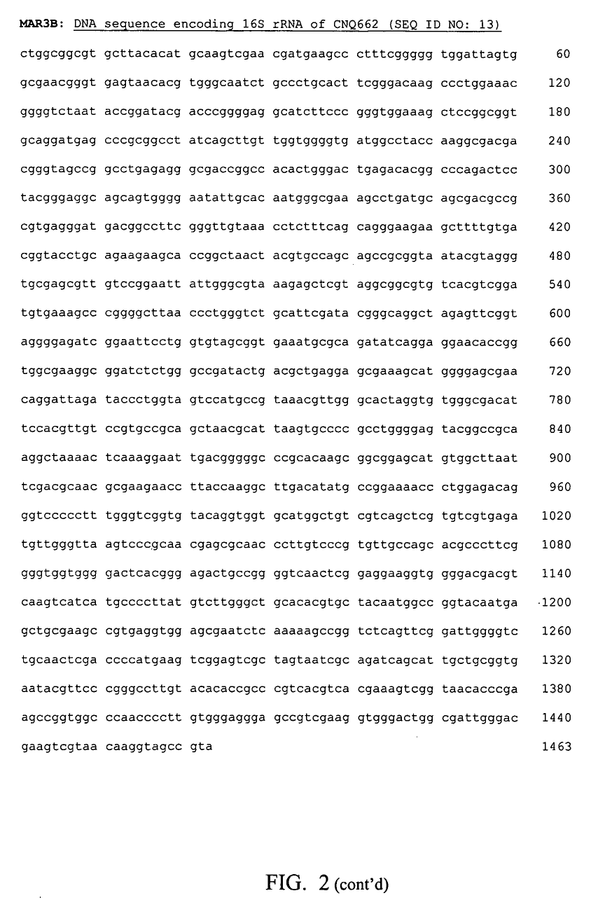

Based on phylogenetic relationships inferred from partial SSU rRNA sequence data, 7 non-“Salinospora” strains were sequenced in full (FIG. 2). One major new clade within Streptomycetaceae has tentatively been called MAR3. The MAR3A strains required seawater for growth. A MAR3 intra-clade similarity of 96.8% suggests that this group is comprised of multiple species.

The tree topology illustrated in FIG. 1 was maintained using multiple treeing methods.

### Example 3

### Production and Isolation of Useful Products

Strains MAR3A and MAR3B were cultured in sea water based medium as described in EXAMPLE 1. For the production of biologically active substances, strains were cultured in liquid A1 medium (1% starch, 0.4% yeast extract, 0.2% peptone, 1.6% agar, 100% sea water) and the whole culture extracted with a suitable chromatographic partitioning agent on an adsorbant resin (XAD-7). The resin was eluted with acetone and the acetone was removed by rotary evaporation. Extracts were solubilized in DMSO and tested for anticancer and antibiotic activities in laboratory assays.

### Example 4

### Antibacterial Assay

Extracts from cultured MAR3A and MAR3B strains were tested using standard methods to demonstrate their antibiotic activity against Gram-positive and Gram-negative bacteria. The method used to test against Staphylococcus aureus is detailed below. Similar methods are used to test for antimicrobial activity against other organisms. Extracts were compared to known antibiotics and relative activity levels determined. Extracts with potent antibiotic activity were further analyzed for the presence of novel metabolites.

Briefly, cultures of S. aureus were grown overnight to stationary phase. The number of bacteria per ml was calculated and a uniform number of bacteria were plated into individual wells containing fresh media. Compounds of interest, including known antibiotic agents (e.g., Oxacillin in DMSO at 0.04 mg/mL), were added to a single row of wells and serially diluted down the plate to determine the concentration required to kill the bacteria. Plates were incubated overnight at 37° C. to allow for cell growth. Samples were read in an automated plate reader at 600 nm and MIC concentrations were determined.

The antibiotic activity for each of the extracts from strains within both phylotypes is provided in the table below.

TABLE 2

Methylicillin Resistant
Staphylococcus (MIC)

MAR3A

| | |
|---|---|
| CNQ687 | 15.6 μg/ml |
| CNQ530 | 15.6 μg/ml |

MAR3B

| | |
|---|---|
| CNR530 | 7.8 μg/ml |
| CNQ265 | 15.6 μg/ml |

### Example 5

### Assay for the Inhibition of Growth of Colon Carcinoma Cells In Vitro

The cytotoxicity of extracts from cells or culture media were assessed in vitro against the human colon carcinoma cell line HCT-116 by MTS assay. Cells were plated at 4,000 cells per well in 96 well microliter plates and, after 24 hours, the extract (dissolved in DMSO or other appropriate solvent) was added and serially diluted. The cells were incubated with the extracts at 37° C. for 72 hours, then the tetrazolium dye MTS was added to a final concentration of 333 μg/ml and the electron coupling agent phenazine methosulfate was added to a final concentration of 25 μM. Once reduced, MTS is converted into a water insoluble blue crystal formazan and that was read at an absorbance at 490 nm with a microplate reader. As dead cells are unable to reduce MTS, the amount of formazan is correlated to the number of viable cells.

The growth inhibition (anticancer) activity for each of the extracts from strains within both phylotypes is provided in the table below.

TABLE 3

% Cell Survival
(HCT-116 Cells)

MAR3A

| | |
|---|---|
| CNQ698 | 4% |

MAR3B

| | |
|---|---|
| CNQ662 | 2% |

### Example 6

### Chemical Mutagenesis of MAR3A and MAR3B, Strains to Generate Overproducing Strains

Chemical mutagenesis of MAR3A and MAR3B strains can be performed to generate strains that overproduce a desired product. For example, a strain that produces an antibiotic at a low level is treated with ethylmethylsulfonate (EMS) during the mid-log growth phase. Mutagenized cultures are streaked onto plates to allow for the isolation of individual clones. From the individual clones, cultures are grown and the antibiotic, in a crude or pure form, is isolated. The relative yields of the compounds of interest produced by the mutagenized strains are compared to the original strain to select an overproducing strain.

### Example 7

### Heterologous Gene Expression

Actinomycete strains have been useful as hosts for the production of secondary metabolites from other more slowly growing organisms (Tang, et al., 2000). Genes, either singly or in clusters, can be expressed in MAR3A and MAR3B strains for the production of proteins or secondary metabolites. Methods for transferring nucleic acids into bacteria are well known by those skilled in the art.

### Example 8

### Gene Cluster Isolation and Expression

The synthesis of a number of actinomycete antibiotics (e.g. actinorhodin, frenolicin, granaticin, griseusin, octatetracycline, and tetracenomycin) are produced by clustered polyketide synthetase (PKS) genes (Hopwood, 1995). PKS genes are classified into two types of large mutifunctional proteins. In PKS type I genes, the substrate progresses through a number of active sites on a single protein. In PKS type II genes, multiprotein complexes are produced and the substrate progresses from one protein to the next. PKS type II genes have been cloned and expressed in heterologous systems, either in their native groupings or in novel combinations. Combining genes for the synthesis of secondary metabolites from MAR3A and MAR3B strains with genes from other actinomycetes provides a novel method of biologically assisted combinatorial chemistry that can lead to the production of novel small molecules. Also, MAR3A and MAR3B biosynthetic genes can be transferred intact or shuffled and expressed in an heterologous host leading to the production of new metabolites. PKS genes are not the only ones that occur in modules. For example, non-ribosomal peptide synthetases are modular as well, and are frequently present in the actinomycetes. Biosynthetic gene clusters from the novel MAR3A and MAR3B groups can be used as genetic feedstock for the expression of novel molecules in heterologous strains or for the over-production of native and recombinant gene products.

### Example 9

### Assay for Anti-Inflammatory Activity

Extracts from MAR3A and MAR3B cultures are tested by measuring inhibition of phorbol-induced inflammation (edema) in a mouse ear assays. This is a conventional test which has been accepted as demonstrating a compound's/extract's effectiveness in reducing inflammation. The compound/extract is topically applied in acetone to the inside pinnae of the ears of mice in a solution containing an edema-causing irritant, i.e., phorbol 12-myristate 13-acetate (PMA). PMA alone (2 microgram per ear) or in combination with varying amounts of the extract is applied to the left ear (5 mice per treatment group) while an acetone (control) is applied to the right. After a 3-hour and 20-minute incubation at 23° C., the mice are sacrificed, the ears removed, and bores taken and weighed. Edema is measured by subtracting the weight of the right ear (control) from the weight of the left ear (treatment). The results are recorded as a percent decrease (inhibition) or percent increase (potentiation) in edema relative to PMA.

### Example 10

### Enzyme Inhibition Assay

Extracts from MAR3A and MAR3B strains could be tested for their ability to inhibit enzyme activity. Extracts could be prepared as described above and serial dilutions of the extract(s) added to enzyme-substrate mixtures to determine an IC₅₀, for the reaction.

### Example 11

### Enzyme Activity Assay

Assays for enzyme activity can be tested by growing MAR3A and MAR3B strains in the presence of substrates of interest including, but not limited to chitin, lignin, cellulose, and other recalcitrant biopolymers, etc. Depending on the substrate, assays can be performed to determine the amount of substrate remaining or the amount of product produced.

### Example 12

### Agriculture/Aquaculture Protection Assay

Assays for the protection of plants from pathogens and general growth enhancement can be performed in a standard greenhouse trial. The strain of interest can be applied to the plant directly or incorporated into the growth media. Plants could be challenged by subjecting them to a pathogen and comparing their growth to control groups treated with a pathogen alone, treated with a MAR3A and MAR3B strain alone, or untreated. Rates of growth could be compared to select for strains with the desired activities.

### REFERENCES

1. Anderson, A. S., Wellington, E. M. H. (2001). The taxonomy of Streptomyces and related genera. Int. J. Syst. Evol. Microbiol. 51:797-814.
2. Blondelle, S. E. and Houghten, R. A. (1996). Novel antimicrobial compounds identified using synthetic combinatorial technology. Trends Biotechnol. 14:60-65.
3. Bull, A. T., Ward, A. C., and Goodfellow, M. (2000). Search and discovery strategies for biotechnology: the paradigm shift. Microbiol. Mol. Biol. Rev. 64:573-606.
4. Cole, J. R., Chai, B., Marsh, T. L., Farris, R. J., Wang, Q., Kulam, S. A., Chandra, S., McGarrell, D. M., Schmidt, T. M., Garrity, G. M., Tiedje, J. M. (2003). The Ribosomal Database Project (RDP-II): previewing a new autoaligner that allows regular updates and the new prokaryotic taxonomy. Nucleic Acids Res. 31:442-443.
5. Colquhoun, J. A., Mexson, J., Goodfellow, M., Ward, A. C., Horikoshi, K., Bull, A. T. (1998). Novel rhodococci and other mycolate actinomycetes from the deep sea. Antonie von Leeuwenhoek 74:27-40.
6. Faulkner, D. J. (2002). Marine natural products. Nat. Prod. Rep. 19:1-48.
7. Feling, R. H., Buchanan, G. O., Mincer, T. J., Kauffman, C. A., Jensen, P. R., Fenical, W. (2003). Salinosporamide A: A highly cytotoxic proteasome inhibitor from a novel microbial source, a marine bacterium of the new genus “Salinospora”. Angew. Chem. Int. Ed. 42:355-357.
8. Goodfellow, M., and Haynes, J. A. (1984). Actinomycetes in marine sediments. In Biological, Biochemical, and Biomedical Aspects of Actinomycetes. Ortiz-Ortiz, L. Bojalil, L. F., and Yakoleff, V. (ed.), New York: Academic Press, pp. 453-472.
9. Helmke, E. and Weyland, H. (1984). Rhodococcus marinonascens sp. nov., an actinomycete from the sea. Int. J. Syst. Bacteriol. 34:127-138.
10. Hopwood, D. A. (1995). Genetic manipulation of Streptomyces polyketide synthase genes for novel secondary metabolite production. FEMS Microbiol. Rev. 16:233-4.
11. Hughes, J. B., Hellmann, J. J., Ricketts, T. H., Bohannan, J. M. (2001). Counting the uncountable: statistical approaches to estimating microbial diversity. Appl. Environ. Microbiol. 67:4399-4406.
12. Jensen, P. R., Dwight, R., Fenical, W. (1991). Distribution of actinomycetes in near-shore tropical marine sediments. Appl. Environ. Microbiol. 57:1102-1108.
13. Jensen, P. R., Fenical, W. (2000). Marine microorganisms and drug discovery: current status and future potential. In Drugs from the Sea. Fusetani, N. (ed.), Basel: S. Karger AG pp. 6-29.
14. Lane D J, Pace B, Olsen G J, Stahl D A, Sogin M L, and Pace N R. (1985). Rapid determination of 16S ribosomal RNA sequences for phylogenetic Analyses. Proc. Natl. Acad. Sci. USA. 82(20):6955-6659.
15. Maddison, D. R., and Maddison, W. P. (2001). MacClade 4. Sinauer Associates, Inc., Sunderland, Mass.
16. Macleod, R. A. (1965). The question of the existence of specific marine bacteria. Bacteriol. Rev. 29:9-23.
17. Mincer, T. J., Jensen, P. R., Kauffman, C. A., and Fenical, W. (2002). Widespread and persistent populations of a major new marine actinomycete taxon in ocean sediments. Appl. Environ. Microbiol. 68:5005-5011.
18. Marmur, J. (1961). A procedure for the isolation of deoxyribonucleic acid from microorganisms J. Mol. Biol. 3:208-218.
19. Moran, M. A., Rutherford, L. T., and Hodson, R. E. (1995). Evidence for indigenous Streptomyces populations in a marine environment determined with a 16S rRNA probe. Appl. Environ. Microbiol. 61:3695-3700.
20. Okami, Y., and Hotta, K. (1988). Search and discovery of new antibiotics. In Actinomycetes in Biotechnology. Goodfellow, M., Williams, S. T., and Mordarski, M. (eds.) New York: Academic Press, pp. 33-67.
21. Pathirana, C., Jensen, P. R., and Fenical W., (1992). Marinone and Debromomarinone: Antibiotic Sequiterpenoid Napthoquinones of a New Structure Class form a Marine Bacterium. Tetrahedron Letters. 33(50):7663-7666.
22. Rappé, M. S. Connon, S. A., Vergin, K. L, Giovannoni, S. (2002). Cultivation of the ubiquitous SAR11 marine bacterioplankton clade. Nature 418:630-633.
23. Rossell-Mora, R., Amann, R. (2001). The species concept for prokaryotes. FEMS Microbiol. Rev. 25:39-67.
24. Sambrook et al. (1989). Molecular Cloning: A Laboratory Manual, 2d Ed., Cold Spring Harbor Laboratory Press, NY.
25. Stach, J. E. M., Maldonado, L. A., Masson, D. G., Ward, A. C., Goodfellow, M., Bull, A. T. (2003). Statistical approaches for estimating actinobcterial diversity in marine sediments. Appl. Environ. Microbiol. 69:6189-6200.
26. Swofford, D. L. (2002). PAUP*. Phylogenetic analysis using parsimony (*and other methods). Version 4. Sinauer Associates, Sunderland, Mass.
27. Takizawa, M., Colwell, R. A., and Hill, R. (1993). Isolation and diversity of actinomycetes in the Chesapeake Bay. Appl. Environ. Microbiol. 59:997-1002.
28. Tang, L., Shah, S., Chung, L., Carney, J., Katz, L., Khosla, C., and Julien, B. (2000). Cloning and heterologous expression of the epothilong gene cluster. Science. 287:640-2.
29. Thompson, J. D., Gibson, T. J., Plewniak, F., Jeanmougin, F., and Higgins, D. G. (1997). The ClustalX windows interface: flexible strategies for multiple sequence alignment aided by quality analysis tools. Nucleic Acids Res. 24:4876-4882.
30. Weyland, H. (1969). Actinomycetes in North Sea and Atlantic Ocean sediments. Nature. 223:858.
31. Weyland, H. (1981). Distribution of actinomycetes on the sea floor. Actinomycetes, Zbl. Bakt. Suppl. 11(Supplemental): 185-193.
32. Wijkmans, J. C. H. M., and Beckett, R. P. (2002). Combinatorial chemistry in anti-infective research. Drug Discovery Today 7:126-132.

Although the invention has been described with reference to the above examples, it will be understood that modifications and variations are encompassed within the spirit and scope of the invention. Accordingly, the invention is limited only by the following claims.

1. A bacterial preparation comprising one or more isolated and purified strain(s) selected from MAR3A or MAR3B which produces one or more bioactive compositions.

2. The bacterial preparation of claim 1, wherein the one or more strains are isolated by selection on media comprising:
i) a solid or liquid medium selective to induce the growth of a marine actinomycete;
ii) seawater;
iii) a nutrient agent to induce the substantial growth of marine actinomycete; and
iv) an antifungal agent.

3. The bacterial preparation of claim 1, wherein the one or more purified strain is MAR3A.

4. The bacterial preparation of claim 1, wherein the one or more purified strain is MAR3B.

5. An isolated bacterium which has a 16S rRNA which is about 98.4% to about 99% homologous to a 16S rRNA comprising SEQ ID NO: 7, SEQ ID NO: 8, SEQ ID NO: 9, or SEQ ID NO: 11.

6. An isolated bacterium which has a 16S rRNA which is about 97.5% to about 99% homologous to a 16S rRNA comprising SEQ ID NO: 10, SEQ ID NO: 12 or SEQ ID NO: 13.

7. The isolated bacteria of any one of claims 5 or 6, having phenotypic characteristics selected from requiring seawater for growth, gram positive, filamentous hyphae, mycelium forming and/or forming leathery colonies that adhere to agar surfaces and any combination thereof.

8. The isolated bacteria of claim 7, selected from bacteria deposited as ATCC Nos. PTA-6061 and PTA-6062.

9. A method of isolating a bioactive composition from a marine actinomycete selected from MAR3A or MAR3B comprising:
i) treating marine samples by:
air-drying and
grinding to form a finely divided particulate;
ii) applying the particulate to a liquid or solid medium that selects for marine actinomycetes;
iii) isolating a culture growing from the medium and/or a supernatant thereof;
iv) extracting the culture and/or the supernatant with an absorbent resin;
v) eluting the extract of step (iv) with a polar organic solvent;
vi) evaporating the solvent; and
vii) solublizing the residue of step (vi) in a chaotropic agent,
wherein the solubilized residue comprises the bioactive composition.

10. The method of claim 9, wherein the medium comprises starch, yeast extract, peptone, and agar.

11. The method of claim 10, wherein the medium comprises up to about 1% starch (w/v).

12. The method of claim 10, wherein the medium comprises up to about 0.4% yeast extract (w/v).

13. The method of claim 10, wherein the medium comprises up to about 0.2% peptone (w/v).

14. The method of claim 10, wherein the media comprises up to about 1.6% agar (w/v).

15. The method of claim 9, wherein the resin is XAD-7.

16. The method of claim 9, wherein the polar organic solvent is acetone.

17. The method of claim 16, wherein the acetone is removed by rotary evaporation.

18. The method of claim 9, wherein the chaotropic agent is DMSO.

19. The method of claim 9, wherein the marine actinomycete is MAR3A.

20. The method of claim 9, wherein the marine actinomycete is MAR3B.

21. An isolated bioactive composition produced by a marine actinomycete selected from MAR3A or MAR3B comprising:
i) culturing the actinomycete from a marine sample, wherein the sample is treated by
air-drying and grinding;
ii) applying the particulate to a liquid or solid medium;
iii) isolating at least one colony from the medium;
iv) growing the colony in a liquid medium;
v) extracting the medium of step (iv) with an absorbent resin;
vi) eluting the extract of step (v) with a polar organic solvent;
vii) evaporating the solvent; and
viii) solublizing the residue of step (vii) in a chaotropic agent,
wherein the solubilized residue comprises the bioactive composition.

22. The isolated bioactive composition of claim 21, wherein the bioactive composition exhibits anticancer or antibiotic activity.

23. The isolated bioactive composition of claim 21, wherein the marine actinomycete is MAR3A.

24. The isolated bioactive composition of claim 21, wherein the marine actinomycete is MAR3B.

25. The isolated bioactive composition of claim 22, wherein the bioactive composition exhibits antibiotic activity.

26. The isolated bioactive composition of claim 25, wherein the bioactive composition is effective against methylicillin-resistant Staphylococcus aureus (MRSA).

27. The isolated bioactive composition of claim 22, wherein the bioactive composition exhibits anticancer activity.

28. The isolated bioactive composition of claim 27, wherein the bioactive composition is effective against HCT-116 cells.

29. An isolated nucleic acid as set forth in SEQ ID NO: 7.

30. An isolated nucleic acid as set forth in SEQ ID NO: 8.

31. An isolated nucleic acid as set forth in SEQ ID NO: 9.

32. An isolated nucleic acid as set forth in SEQ ID NO: 10.

33. An isolated nucleic acid as set forth in SEQ ID NO: 11.

34. An isolated nucleic acid as set forth in SEQ ID NO: 12.

35. An isolated nucleic acid as set forth in SEQ ID NO: 13.

36. The isolated nucleic acid of any one of claims 29-35, wherein the nucleic acid is DNA or RNA.

37. A vector comprising the nucleic acid of claim 36.

38. A host cell comprising the vector of claim 37.

39. A method for drug discovery comprising growing a strain of actinomycete selected from MAR3A or MAR3B in a growth medium, collecting the actinomycete or conditioned growth medium, and analyzing the actinomycete or conditioned medium for pharmacological activity.

40. The method of claim 39, wherein analyzing comprises an assay for antibiotic activity.

41. The method of claim 39, wherein analyzing comprises an assay for anti-cancer activity.

42. A method of treating cancer or bacterial infection by administering a therapeutically effective amount of a pharmaceutical composition comprising a bioactive composition from a marine actinomycete selected from MAR3A or MAR3B to a subject in need thereof.

43. The method of claim 42, wherein the bioactive composition is isolated from MAR3A.

44. The method of claim 42, wherein the bioactive composition is isolated from MAR3B.

45. A pharmaceutical composition comprising a bioactive composition from a marine actinomycete selected from MAR3A or MAR3B and a pharmaceutically acceptable carrier.

46. The pharmaceutical composition of claim 45, wherein the bioactive composition is isolated from MAR3A.

47. The pharmaceutical composition of claim 45, wherein the bioactive composition is isolated from MAR3B.

48. A method for identifying a clade member for a bacterial strain comprising:
a) performing a nucleic acid BLAST comparison search of a database using default parameters by inputting a 16S rRNA sequence of the bacterial strain into a sequence comparison interface of the database;
b) analyzing a text file comprising a subset of resulting hits on a ribosomal database by uploading the text file into a sequence matching interface on the ribosomal database; and
c) determining sequence homology between the subset of hits from the BLAST search and sequences for MAR3A or MAR3B strains of bacteria,
wherein a strain is identified as belonging to the MAR3A or MAR3B clade based on the similarity between sequence alignments that are generated by the sequence matching interface of the ribosomal database.

49. The method of claim 48, wherein the subset comprises sequences from strains of Streptomycetaceae.

50. The method of claim 48, wherein the database of step (a) is the NCBI BLAST database.

51. The method of claim 50, wherein the inputted sequence is entered into a dialog box/browser using nr as the default value for conducting the search in the database of step (a).

52. The method of claim 48, wherein the text file is in FASTA format.

53. The method of claim 48, wherein the interface of step (c) is a PHYLIP interface.

54. The method of claim 48, wherein step (c) further comprises distance matrix analysis.

55. The method of claim 48, further comprising generating a phylogenetic tree.

56. The method of claim 48, wherein the sequences for MAR3A are selected from SEQ ID NO: 7, SEQ ID NO: 8, SEQ ID NO: 9, or SEQ ID NO: 11.

57. The method of claim 56, wherein similarity between sequence alignments are greater than or equal about 98.5%.

58. The method of claim 48, wherein the sequences for MARB3 comprise nucleotides U, G, C, and A/C at E. coli 16S rRNA alignment positions 145, 771, 808 and 1005, respectively.

59. The method of claim 58, wherein the sequences for MAR3B are selected from SEQ ID NO: 10, SEQ ID NO: 12 or SEQ ID NO: 13.

60. The method of claim 59, wherein similarity between sequence alignments are greater than or equal to about 97.5%.

61. A strain identified by the method of claim 48, wherein the strain belongs to the MAR3A or MAR3B group.


SEQUENCE LISTING (13 sequences)

SEQ ID NO: 1 - DNA, 20 bp, Artificial sequence
  Amplification primer
agagtttgatcctggctcag

SEQ ID NO: 2 - DNA, 22 bp, Artificial sequence
  Amplification primer
tacggctaccttgttacgactt

SEQ ID NO: 3 - DNA, 19 bp, Artificial sequence
  Amplification primer
gtgccagcagccgcggtaa

SEQ ID NO: 4 - DNA, 16 bp, Artificial sequence
  Amplification primer
gcaacgagcgcaaccc

SEQ ID NO: 5 - DNA, 19 bp, Artificial sequence
  Amplification primer
ccgcggctgctggcacgta

SEQ ID NO: 6 - DNA, 19 bp, Artificial sequence
  Amplification primer
gtgcgggcccccgtcaatt

SEQ ID NO: 7 - DNA, 1467 bp, Actinomycete
  misc_feature (1)..(1467): n is any nucleotide
tggcggcgtgcttacacatgcaagtcgaacgatgaagccgcttcggtggtggattagtggcgaacgggtgagtaacacgtgggcaatctgccctgcactctgggacaagccctggaaacggggtctaataccggatatgatcaccggccgcatggtctggtggtggaaagctccggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagctttcgggtgacggtacctgcagaagaagcaccggctaactacgtgccagcagccgcggtaatacgtagggtgcgagcgttgtccggaattattgggcgtaaagagctcgtaggcggcctgtcgcgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggcactaggtgtgggccgcattccacgtggtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaacncccngagangcttgcccccttgtggtcggtgtacaggtggtgcatggctgtcgtcagctcgtgtcgtgagatgttgggttaagtcccgcaacgagcgcaacccttatcctgtgttgccagcacgcccttcggggtggtggggactcacgggagactgccggggtcaactcggaggaaggtggggacgacgtcaagtcatcatgccccttatgtcttgggctgcacacgtgctacaatggccggtacaatgagttgcgatgccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaacccccttgtggggagggaatcgtcgaaggtgggactggcgattgggacgaagtcgtaacaagggtagccgt

SEQ ID NO: 8 - DNA, 1322 bp, Actinomycete
gtggtggattagtggcgaacgggtgagtaacacgtgggcaatctgccctgcactctgggacaagccctggaaacggggtctaataccggatatgatcaccggccgcatggtctggtggtggaaagctccggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagctttcgggtgacggtacctgcagaagaagcaccggctaactacgtgccagcagccgcggtaatacgtagggtgcgagcgttgtccggaattattgggcgtaaagagctcgtaggcggcctgtcgcgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggcactaggtgtgggccgcattccacgtggtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaacacgtggagagacctgcccccttgtggtcggtgtacaggtggtgcatggctgtcgtcagctcgtgtcgtgagatgttgggttaagtcccgcaacgagcgcaacccttatcctgtgttgccagcacgcccttcggggtggtggggactcacgggagactgccggggtcaactcggaggaaggtggggacgacgtcaagtcatcatgccccttatgtcttgggctgcacacgtgctacaatggccggtacaatgagttgcgatgccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaa

SEQ ID NO: 9 - DNA, 1497 bp, Actinomycete
  misc_feature (990)..(990): n is any nucleotide
agagtttgatcctggctcaggacgaacgctggcggcgtgcttaacacatgcaagtcgaacgatgaagccgcttcggtggtggattagtggcgaacgggtgagtaacacgtgggcaatctgccctgcactctgggacaagccctggaaacggggtctaataccggatatgatcaccggccgcatggtctggtggtggaaagctccggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgcaagcctgatgcagcgacgccgcgtgagggatgacggccttcgggttgtaaacctctttcagcagggaagaagctttcgggtgacggtacctgcagaagaagcaccggctaactacgtgccagcagccgcggtaatacgtagggtgcgagcgttgtccggaattattgggcgtaaagagctcgtaggcggcctgtcgcgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggcactaggtgtgggccgcattccacgtggtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaacacgtggaganacgtgcccccttgtggtcggtgtacaggtggtgcatggctgtcgtcagctcgtgtcgtgagatgttgggttaagtcccgcaacgagcgcaacccttatcctgtgttgccagcacgcccttcggggtggtggggactcacgggagactgccggggtcaactcggaggaaggtggggacgacgtcaagtcatcatgccccttatgtcttgggctgcacacgtgctacaatggccggtacaatgagttgcgatgccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaacccccttgtggggagggaatcgtcgaaggtgggactggcgattgggacgaagtcgtaacaaggtagccgta

SEQ ID NO: 10 - DNA, 1405 bp, Actinomycete
  misc_feature (933)..(934): n is any nucleotide
catgcaagtcgaacgatgaagctccttcgggggtggattagtggcgaacgggtgagtaacacgtgggcaatctgccctgtacttcgggacaagccctggaaacggggtctaataccggatgggactgccggccgcatggtctggtggtggaaagcttcggcggtacaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcggaagcctgatgcagcgacgccgcgtgagggatgacggccttcgggttgtaaacctctttcagcagggaagaagcctttaaagtgacggtacctgcagaagaagcaccggctaactacgtgccagcagccgcggtaatacgtagggtgcgagcgttgtccggaattattgggcgtaaagagctcgtaggcggcctgtcgcgtcggatgtgaaagcccggggcttaaccctgggtctgcattcgatacgggcaggctggagttcggtaggggagatcggaattcctggtgtagcggtgaaatgcgcagatatcaggaggaacaccggtggcgaaggcggatctctgggccgatactgacgctgaggagcgaaagcatggggagcgaacaggattagataccctggtagtccatgccgtaaacgttgggcactaggtgtgggcgacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccgganngccgcagagatgtggtttcccttgtggtcggtgtacaggtggtgcatggctgtcgtcagctcgtgtcgtgagatgttgggttaagtcccgcaacgagcgcaacccttgtctcgtgttgccagcaggcccttgtggtgctggggactcacgggagactgccggggtcaactcggaggaaggtggggacgacgtcaagtcatcatgccccttatgtcttgggctgcacacgtgctacaatggccggtacaatgagtggcgatgccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggagccgtcgaa

SEQ ID NO: 11 - DNA, 1486 bp, Actinomycete
cctggctcaggacgaacgctggcggcgtgcttaacacatgcaagtcgaacgatgaagccgcttcggtggtggattagtggcgaacgggtgagtaacacgtgggcaatctgccctgcactctgggacaagccctggaaacggggtctaataccggatacgaccaccggccgcatggtctggtggtggaaagctccggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagctttcgggtgacggtacctgcagaagaagcaccggctaactacgtgccagcagccgcggtaatacgtagggtgcgagcgttgtccggaattattgggcgtaaagagctcgtaggcggcctgtcgcgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcaacattccacgttgtccgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttatcctgtgttgccagcatgcccttcggggtgatggggactcacgggagactgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggaatcgtcgaaggtgggactggcgattgggacgaagtcgtaacaagggtagccgta

SEQ ID NO: 12 - DNA, 1497 bp, Actinomycete
tagagtttgatccctggctcaggacgaacgctggcggcgtgcttaacacatgcaagtcgaacgatgaagctccttcgggggtggattagtggcgaacgggtgagtaacacgtgggcaatctgccctgcacttcgggacaagccctggaaacggggtctaataccggatgggactgccggccgcatggtctggtggtggaaagcttcggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcggaagcctgatgcagcgacgccgcgtgagggatgacggccttcgggttgtaaacctctttcagcagggaagaagcctttaaagtgacggtacctgcagaagaagcaccggctaactacgtgccagcagccgcggtaatacgtagggtgcgagcgttgtccggaattattgggcgtaaagagctcgtaggcggcctgtcgcgtcggatgtgaaagcccggggcttaaccccgggtctgcattcgatacgggcaggctggagttcggtaggggagatcggaattcctggtgtagcggtgaaatgcgcagatatcaggaggaacaccggtggcgaaggcggatctctgggccgatactgacgctgaggagcgaaagcatggggagcgaacaggattagataccctggtagtccatgccgtaaacgttgggcactaggtgtgggcgacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggattgccgcagagatgcggtttcccttgtggtcggtgtacaggtggtgcatggctgtcgtcagctcgtgtcgtgagatgttgggttaagtcccgcaacgagcgcaacccttatctcgtgttgccagcaggcccttgtggtgctggggactcacgggagactgccggggtcaactcggaggaaggtggggacgacgtcaagtcatcatgccccttatgtcttgggctgcacacgtgctacaatggccggtacaatgagttgcgaatccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggagccgtcgaaggtgggactggcgattgggacgaagtcgtaacaaggtagccgta

SEQ ID NO: 13 - DNA, 1463 bp, Actinomycete
ctggcggcgtgcttacacatgcaagtcgaacgatgaagccctttcgggggtggattagtggcgaacgggtgagtaacacgtgggcaatctgccctgcacttcgggacaagccctggaaacggggtctaataccggatacgacccggggaggcatcttcccgggtggaaagctccggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcttttgtgacggtacctgcagaagaagcaccggctaactacgtgccagcagccgcggtaatacgtagggtgcgagcgttgtccggaattattgggcgtaaagagctcgtaggcggcgtgtcacgtcggatgtgaaagcccggggcttaaccctgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcatggggagcgaacaggattagataccctggtagtccatgccgtaaacgttgggcactaggtgtgggcgacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatatgccggaaaaccctggagacagggtccccctttgggtcggtgtacaggtggtgcatggctgtcgtcagctcgtgtcgtgagatgttgggttaagtcccgcaacgagcgcaacccttgtcccgtgttgccagcacgcccttcggggtggtggggactcacgggagactgccggggtcaactcggaggaaggtggggacgacgtcaagtcatcatgccccttatgtcttgggctgcacacgtgctacaatggccggtacaatgagctgcgaagccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggagccgtcgaaggtgggactggcgattgggacgaagtcgtaacaaggtagccgta